(19) United States
(12) Patent Application Publication
(10) Pub. No.: US 2020/0205724 A1
(43) Pub. Date: Jul. 2, 2020

(54) ELECTRONIC DEVICE AND STRESS MEASUREMENT METHOD THEREOF

(71) Applicant: Samsung Electronics Co., Ltd., Suwon-si, Gyeonggi-do (KR)

(72) Inventors: Donghyun LEE, Seoul (KR); Seunghwan SHIN, Seongnam-si (KR); Junseok OH, Suwon-si (KR); Jongmin CHOI, Seoul (KR); Jinwoo SEO, Seoul (KR); Seung-Eun LEE, Seoul (KR)

(21) Appl. No.: 16/640,926

(22) PCT Filed: Jun. 21, 2018
(86) PCT No.: PCT/KR2018/007011

§ 371 Date: Feb. 21, 2020

(30) Foreign Application Priority Data
Aug. 22, 2017 (KR) 10-2017-0106277

(51) Int. Cl. A61B 5/00; A61B 5/024; A61B 5/0205

(52) U.S. Cl. CPC A61B 5/486; A61B 5/024; A61B 5/0205; A61B 5/7278; A61B 5/742; A61B 5/4884

(57) ABSTRACT

Various embodiments of the present invention relate to an electronic device and a stress measurement method thereof. The electronic device comprises: a memory for storing a first histogram for determining reference information for computing health condition of a user; a biometric sensor; and at least one processor functionally connected to the memory and the biometric sensor, wherein the at least one processor may be configured to acquire biometric information via the biometric sensor, generate a second histogram by analyzing the acquired biometric information, accumulate the second histogram in the first histogram, thereby updating the first histogram, and update the reference information on the basis of the updated first histogram. Other various embodiments are possible.

### TECHNICAL FIELD

Various embodiments of the disclosure relate to an electronic device and a stress measurement method thereof.

### BACKGROUND ART

Electronic devices (for example, mobile terminals, smartphones, wearable electronic devices, or the like) may provide various functions. For example, a smartphone may provide short-range wireless communication (for example, Bluetooth, wireless fidelity, near field communication (NFC), or the like), mobile communication (3^{rd} generation (3G), 4G, 5G, or the like), a music or video replay function, a capturing function, a navigation function, a messenger function, etc., in addition to a basic voice communication function.

The electronic devices may provide a variety of health-related information. For example, recent electronic devices may provide stress information. In general, the electronic devices may measure a stress based on a heart rate variability.

### DISCLOSURE OF INVENTION

### Technical Problem

Cardiovascular characteristics vary from user to user, and user's cardiovascular characteristics may change with time. However, a related-art electronic device equally applies reference information for calculating a stress to all users. In addition, the related-art electronic device may measure a stress by using fixed reference information without considering a change in the cardiovascular characteristics over time. The related-art electronic device may have a problem since it measures a stress without considering users' respective cardiovascular characteristics and changes in the cardiovascular characteristics over time as described above. That is, the related-art electronic device may not provide an exact stress measurement result.

Various embodiments of the disclosure to solve the above-described problems provide an electronic device capable of updating reference information (baseline) for measuring a stress and a stress measurement method thereof.

### Solution to Problem

According to various embodiments of the disclosure, an electronic device may include: a memory configured to store a first histogram which is used to determine reference information for calculating a health condition of a user; a biometric sensor; and at least one processor operatively connected with the memory and the biometric sensor, and the at least one processor may be configured to: obtain biometric information through the biometric sensor; generate a second histogram by analyzing the obtained biometric information; update the first histogram by accumulating the second histogram in the first histogram; and update the reference information based on the updated first histogram.

According to various embodiments of the disclosure, a stress measurement method of an electronic device which stores a first histogram which is used to determine reference information for calculating a health condition of a user may include: obtaining biometric information through a biometric sensor; generating a second histogram by analyzing the obtained biometric information; updating the first histogram by accumulating the second histogram in the first histogram; and updating the reference information based on the updated first histogram.

According to various embodiments of the disclosure, an electronic device may include: a display; a biometric sensor; and a processor operatively connected with the display and the biometric sensor, and the processor may be configured to: obtain one or more pieces of first biometric information through the biometric sensor; determine a numerical value related to a user's stress by using the one or more pieces of first biometric information, based at least on a first histogram which is generated according to a frequency corresponding to a change of a period during which one or more pieces of second biometric information obtained before the one or more pieces of first biometric information are obtained are measured, and, based at least on the determination, generate a second histogram in which a frequency corresponding to a change of a period during which the one or more pieces of first biometric information are measured is accumulated, to determine another numerical value related to the user's stress by using one or more pieces of third biometric information to be measured from the user based at least on the second histogram.

### Advantageous Effects of Invention

Various embodiments of the disclosure can continuously update reference information according to a change in cardiovascular characteristics of a user, and can exactly measure and provide a stress. Various embodiments of the disclosure described above can exactly provide a stress by reflecting a change in cardiovascular characteristics of a user, and can enhance reliability of the user regarding a stress measurement result.

In addition, various embodiments of the disclosure can share reference information among a plurality of electronic devices, and can prevent a problem that measurement results are different according to electronic devices due to different reference information of the respective electronic devices.

In addition, various embodiments of the disclosure can prevent a measurement deviation caused by a difference in performance of heart rate measurement sensors for measuring a heart rate variability (HRV). For example, when a user owns a plurality of electronic devices, measurement results of heart rate measurement sensors (for example, heart rate measurement sensors of relatively low performance) included in other electronic devices may be interpolated with reference to a heart rate measurement sensor of the highest performance (or a heart rate measurement sensor of an electronic device designated by the user), and the plurality of electronic devices can provide similar (substantially the same) measurement results.

### BRIEF DESCRIPTION OF DRAWINGS

FIG. 1 is a block diagram of an electronic device in a network environment according to various embodiments of the disclosure;

FIG. 2 is a view illustrating an example of an electronic device capable of measuring a stress according to an embodiment of the disclosure;

FIG. 3 is a block diagram illustrating a configuration of an electronic device according to an embodiment of the disclosure;

FIGS. 4A, 4B, and 4C are views to explain a stress calculation method of a stress calculation module of FIG. 3;

FIG. 5 is a view to explain a method for updating reference information of a reference information management module of FIG. 3;

FIG. 6 is a view illustrating an example of a user interface providing a stress measurement result of an electronic device according to an embodiment of the disclosure;

FIG. 7 is a view to explain a method for sharing a reference information histogram according to an embodiment of the disclosure;

FIG. 8 is a view to explain a method for interpolating a heart rate measurement result according to an embodiment of the disclosure;

FIG. 9 is a sequence diagram to explain a method for generating a reference information histogram of an electronic device according to an embodiment of the disclosure;

FIG. 10A is a sequence diagram to explain a method for updating reference information according to an embodiment of the disclosure;

FIG. 10B is a sequence diagram to explain the method for updating the reference information according to an embodiment of the disclosure;

FIG. 11 is a sequence diagram to explain a stress measurement method of an electronic device according to an embodiment of the disclosure; and

FIG. 12 is a view illustrating an example of stress measurement of an electronic device according to an embodiment of the disclosure.

### BEST MODE FOR CARRYING OUT THE INVENTION

Hereinafter, various embodiments of the disclosure will be described with reference to the accompanying drawings. In the disclosure, specific embodiments are illustrated in the drawings and relevant detailed descriptions are provided, but this are not intended to limit various embodiments of the disclosure to specific forms. For example, it is obvious to those skilled in the art to which the disclosure belongs that embodiments of the disclosure can be variously changed.

Prior to explaining in detail, an example of measuring a stress based on a heart rate variability (HRV), and updating and sharing reference information for calculating the stress will be described hereinafter. However, embodiments of the disclosure can continuously update a variety of reference information for determining a health condition of a user by considering (reflecting) a change in biological characteristics of the user over time, and may share the updated reference information with another electronic device and/or a server.

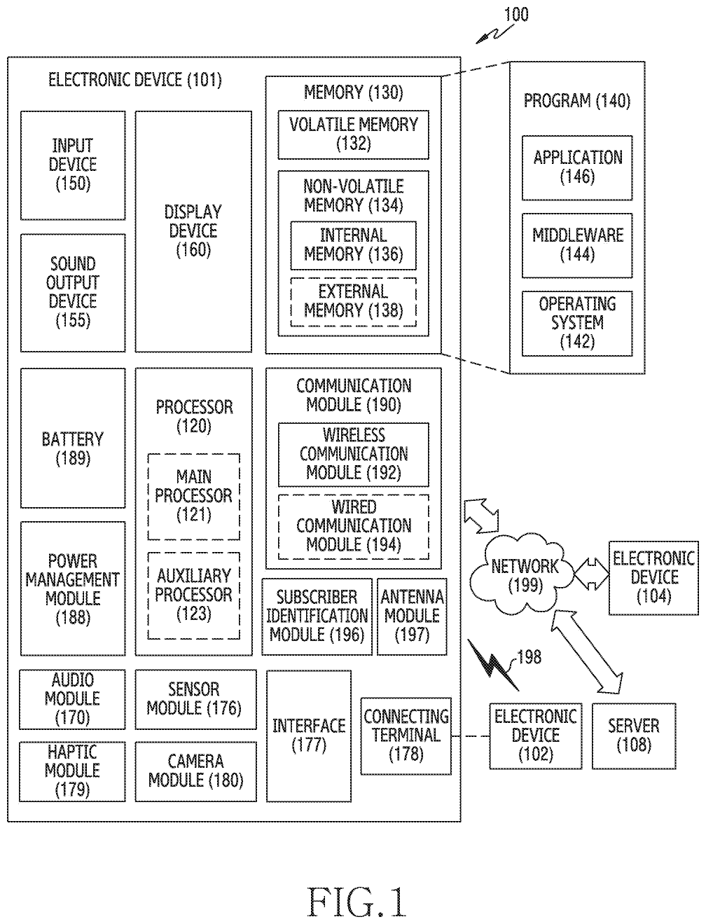

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication), or an electronic device 104 or a server 108 via a second network 199 (e.g., a long-range wireless communication). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, and an antenna module 197. In some embodiments, at least one (e.g., the display device 160 or the camera module 180) of the components may be omitted from the electronic device 101, or other components may be added in the electronic device 101. In some embodiments, some of the components, for example, may be integrated and implemented such as the sensor module 176 (e.g., a fingerprint sensor, an iris sensor, or an illuminance sensor) embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing and computation. The processor 120 may load and process a command or data received from another component (e.g., the sensor module 176 or the communication module 190) in volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from the main processor 121, may additionally or alternatively consume less power than the main processor 121, or to be specific to a specified function. The auxiliary processor 123 may be implemented as separate from, or embedded in the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., performing an application). According to an embodiment, the auxiliary processor 123 (e.g., an image signal processor or a communication processor) may be implemented as part of another component (e.g., the camera module 180 or the communication module 190) functionally related to the auxiliary processor 123. The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, or a keyboard.

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to a user of the electronic device 101. The display device 160 may include, for example, a display, a hologram device, or a projector and control circuitry to control a corresponding one of the display, hologram device, and projector. According to an embodiment, the display device 160 may include touch circuitry, or a pressure sensor adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or an external electronic device (e.g., an electronic device 102 (e.g., a speaker or a headphone)) wiredly or wirelessly coupled with the electronic device 101.

The sensor module 176 may generate an electrical signal or data value corresponding to an operational state (e.g., power or temperature) of the electronic device 101 or an environmental state external to the electronic device 101. The sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support a specified protocol for coupling with the external electronic device (e.g., the electronic device 102) wiredly or wirelessly. According to an embodiment, the interface 177 may include a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). The connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. The haptic module 179 may include, for example, a motor, a piezoelectric element, or an electric stimulator.

The camera module 180 may capture a still image or moving images. According to an embodiment, the camera module 180 may include one or more lense, image sensor, image signal processor, or flash.

The power management module 188 may manage power supplied to the electronic device 101. The power management module 188 may be implemented as at least part of, for example, a power management integrated circuit (PMIC).

The battery 189 may supply power to at least one component of the electronic device 101. The battery 189 may include, for example, a primary cell which is not rechargeable, a secondary cell which is rechargeable, or a fuel cell.

The communication module 190 may support establishing a wired communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a wired communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single chip, or may be implemented as multi chips separate from each other.

The wireless communication module 192 may identify and authenticate the electronic device 101 in a communication network using subscriber information stored in the subscriber identification module 196.

The antenna module 197 may include one or more antennas to transmit or receive a signal or power to or from the outside. According to an embodiment, the communication module 190 (e.g., the wireless communication module 192) may transmit or receive the signal to or from the external electronic device through an antenna appropriate for a communication scheme.

Some of the above-described components may be coupled mutually and communicate signals (e.g., commands or data) therebetween via an inter-peripheral communication scheme (e.g., a bus, general purpose input/output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices. According to an embodiment, if the electronic device 101 should perform a function or a service automatically, or by a request, the electronic device 101, instead of, or in addition to, executing the function or the service, may request at least part associated with the function or the service to external electronic devices. The external electronic devices receiving the request may perform the function requested, or an additional function, and transfer an outcome of the performing to the electronic device 101. The electronic device 101 may provide the outcome, with or without further processing of the outcome. To that end, a cloud computing, distributed computing, or client-server computing technology may be used, for example.

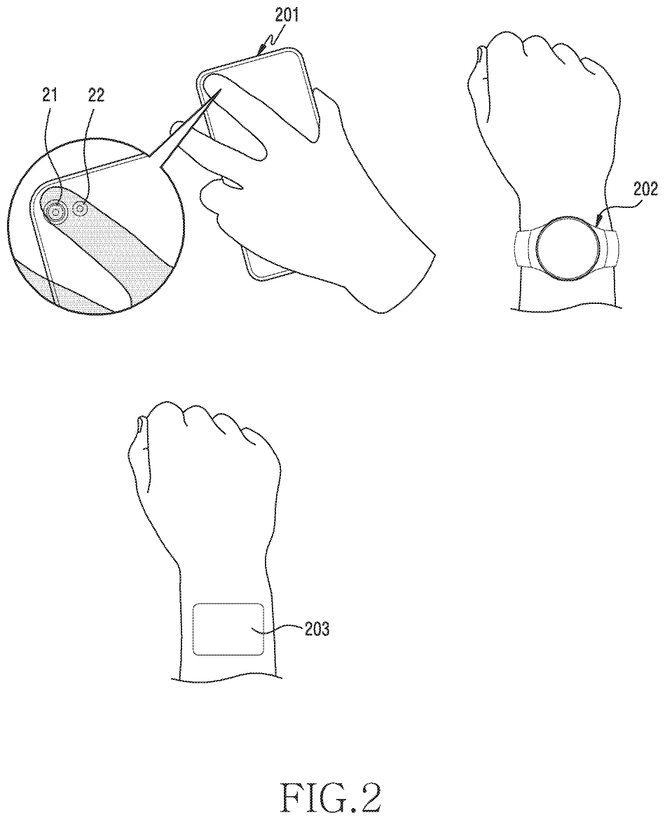

FIG. 2 is a view illustrating an example of an electronic device capable of measuring a stress according to an embodiment of the disclosure.

Referring to FIG. 2, the electronic device according to an embodiment of the disclosure may have various shapes. For example, the electronic device may be a portable electronic device 201 such as a smart phone, a tablet personal computer (PC), or the like, a wearable electronic device (for example, a watch, ring, bracelet, anklet, or necklace-shaped electronic device) 202 which is wearable on a part of user's body, or a body-mounted electronic device (for example, a skin pad or tattoos) 203 which can be attached to a part of user's body. According to an embodiment, the electronic device may be a bio-implantable electronic device which can be inserted into the body.

The portable electronic device 201 may measure an HRV by using a camera 21 and a flash 22. For example, when a user covers the camera 21 and the flash 22 with user's finger (for example, index finger), light of the flash 22 may enter the camera 21 through the finger, and the portable electronic device 201 may measure a heart rate by analyzing a change in the light entering the camera 21. This uses the principle that an amount of light entering the camera 21 is changed according to a change the blood flow of the finger caused by the heart rate. Alternatively, the portable electronic device 201 may measure the heart rate through a separate biometric sensor (for example, a heart rate measurement sensor).

According to an embodiment of the disclosure, the heart rate measurement sensor may include a heart rate sensor for measuring a heart rate by using infrared rays.

The wearable electronic device 202 may include a biometric sensor (for example, a heart rate measurement sensor) disposed on a portion (for example, a bottom surface) contacting user's skin. The body-mounted electronic device 203 may be attached to a part of the body (for example, arm, wrist, leg, neck, head, or the like) to measure an HRV.

According to an embodiment, the wearable electronic device 202 and the body-mounted electronic device 203 may be wirelessly or wiredly connected with an external electronic device (for example, the portable electronic device 201) or a server (not shown) to transmit a measurement result to the external electronic device.

The electronic devices 201, 202, 203 according to an embodiment of the disclosure may measure an HRV upon receiving a user's request or periodically. For example, the portable electronic device 201 may measure the HRV according to a user request, and the wearable electronic device 202 or the body-mounted electronic device 203 may measure the HRV upon receiving a user's request or at set intervals.

The electronic devices 201, 202, 203 may calculate a health condition (for example, a stress) of the user based on the HRV. For example, the electronic devices 201, 202, 203 may calculate the stress by using a personalized-pNNx algorithm Herein, the pNNx algorithm may measure the stress based on reference information (baseline). For example, the pNN50 may have reference information of 50 ms, and may measure a stress according to a ratio of the number of intervals (for example, N-N interval or R-R interval) between continuous heart rates that have a difference exceeding 50 ms, and the total number of intervals between heart rates. The personalized-pNNx may have reference information that is set differently according to a user. For example, the personalized-pNNx may determine reference information based on heart rate data measured in a resting state of the user. The reference information may be determined by a median.

The electronic devices 201, 202, 203 according to an embodiment of the disclosure may periodically measure an HRV, and may update the reference information by reflecting a measurement result.

According to an embodiment, the electronic devices 201, 202, 203 may share the reference information with at least one external electronic device or a server. Herein, the at least one external electronic device may be an electronic device of the same user. This will be described in detail hereinbelow.

According to an embodiment, the electronic devices 201, 202, 203 may calibrate measured HRV data by using interpolation. This will be described in detail hereinbelow.

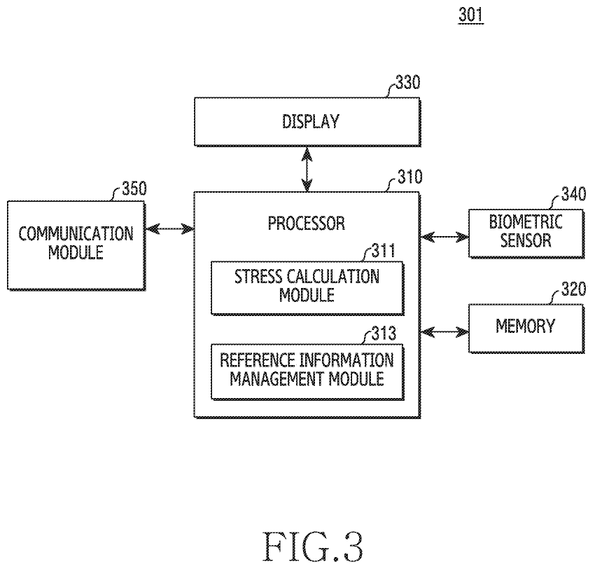

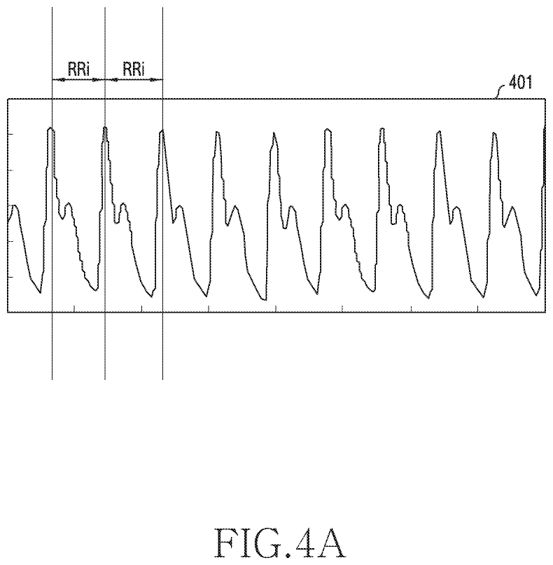

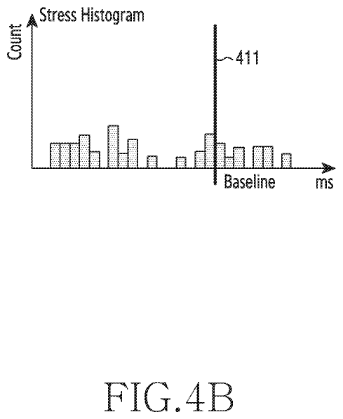

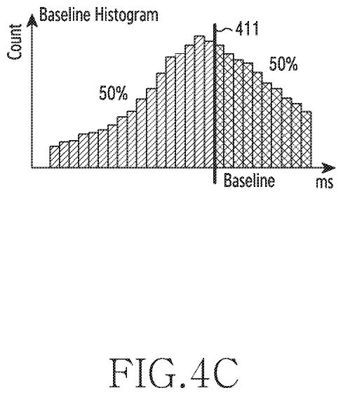

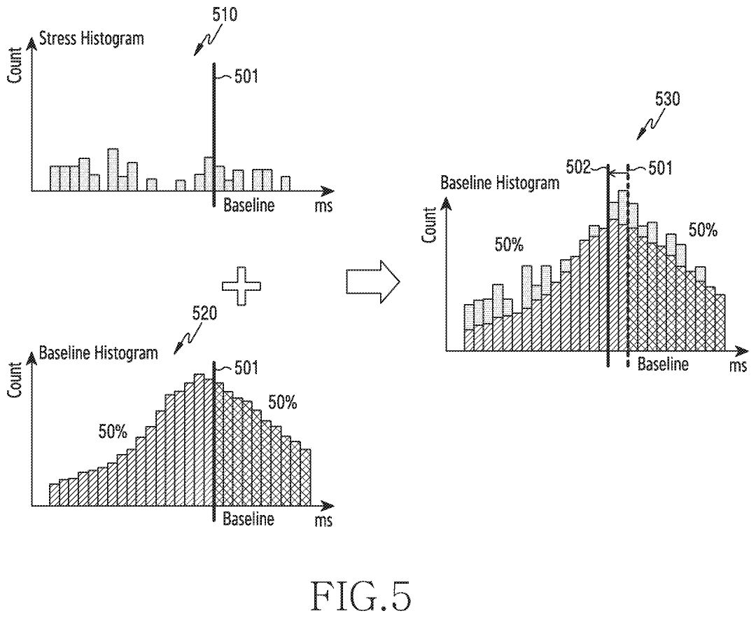

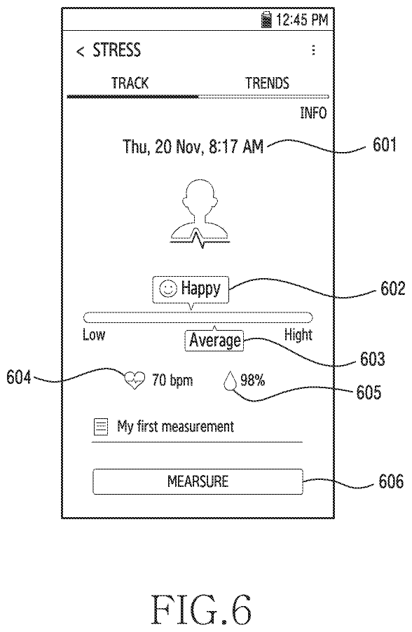

FIG. 3 is a block diagram illustrating a configuration of an electronic device according to an embodiment of the disclosure, FIGS. 4A, 4B, and 4C are views to explain a stress calculation method of a stress calculation module of FIG. 3, FIG. 5 is a view to explain a method for updating reference information of a reference information management module of FIG. 3, and FIG. 6 is a view illustrating an example of a user interface providing a stress measurement result of an electronic device according to an embodiment of the disclosure.

Referring to FIGS. 3 to 6, the electronic device 301 according to an embodiment of the disclosure may include, for example, an entirety or a part of the electronic device 100 shown in FIG. 1.

The electronic device 301 according to an embodiment of the disclosure may include a processor 310, a memory 320, a display 330, a biometric sensor 340, and a communication module 350.

The processor 310 (for example, the processor 120) may control overall operations of the electronic device 301. For example, the processor 210 may control the respective components of the electronic device 301. The processor 310 may receive a command or instructions from the memory 320, and may control the respective components according to the received command or instructions to perform various functions.

The processor 310 may be formed with a central processing unit (CPU), an application processor (AP), a micro control unit (MCU), a micro processor unit (MPU), or the like. The processor 310 may be formed with a single core processor or a multi-core processor. According to another embodiment, the processor 320 may be a multi-processor formed with a plurality of processors. For example, the processor 310 may include an application processor and a communication processor (CP).

The processor 310 according to an embodiment of the disclosure may generate a reference information histogram based on HRV data which is measured through the biometric sensor (for example, a heart rate measurement sensor) 340 in the resting state of the user. The processor 310 may calculate a stress based on the HRV measured through the biometric sensor 340. The processor 310 may update the reference information histogram which serves as a criterion for calculating a stress. The processor 310 may include a stress calculation module 311 and a reference information management module 313.

The stress calculation module 311 may generate a stress histogram based on the HRV measured through the biometric sensor 340. For example, the stress calculation module 311 may calculate intervals (R-R interval: RRi) between an R pulse and an R pulse from a graph 401 indicating a heart rate periodic change as shown in FIG. 4A, calculate a difference of the intervals RRi (delta RRi: dRRi), and may generate a first histogram (for example, a stress histogram) based on the calculated differences as shown in FIG. 4B.

The stress calculation module 311 may calculate the stress based on a ratio of the total number of dRRi and the number of dRRi positioned below predesignated reference information (baseline) 411. For example, when the total number of dRRi is 100 and the number of dRRi having values below the reference information is 60, a value related to the stress may be 60.

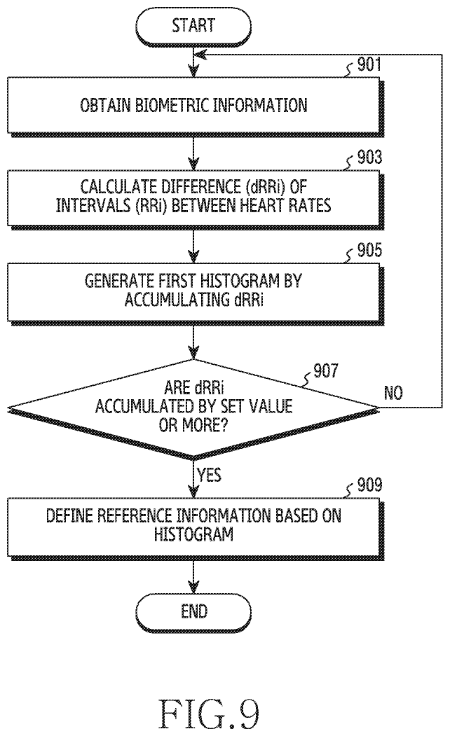

Herein, the reference information may be a value that may be changed according to user's cardiovascular characteristics, and may be set based on a reference information histogram which is generated in the resting state of the user. For example, the reference information 411 may be set based on a median of the reference information histogram as shown in FIG. 4C. For example, when the total number of dRRi is 101, the reference information may be a value of dRRi of the 51st size. Alternatively, when the number of dRRi is 100, the reference information may be an average of a value of dRRi of the 50th size and a value of dRRi of the 51st size. A method for determining the reference information will be described in detail with reference to FIG. 9.

The reference information management module 313 may control updating of the reference information histogram. For example, the reference information management module 313 may generate an updated reference information histogram 530 by adding a pre-stored reference information histogram 520 to a currently measured stress histogram 510. In response to the reference information histogram 520 being updated, the reference information management module 313 may update the reference information to a median of the updated reference information histogram 530. For example, as shown in FIG. 5, the reference information management module 313 may change first reference information 501 to second reference information 502.

According to an embodiment, the reference information management module 313 may update the reference information histogram by multiplying the reference information histogram by a forgetting factor and then accumulating the stress histogram. The forgetting factor may vary in proportion to time. For example, the forgetting factor may vary in proportion to a difference between a current time at which the stress histogram is measured and the most recent measurement time. This is because old data is not likely to be related to current cardiovascular characteristics of the user.

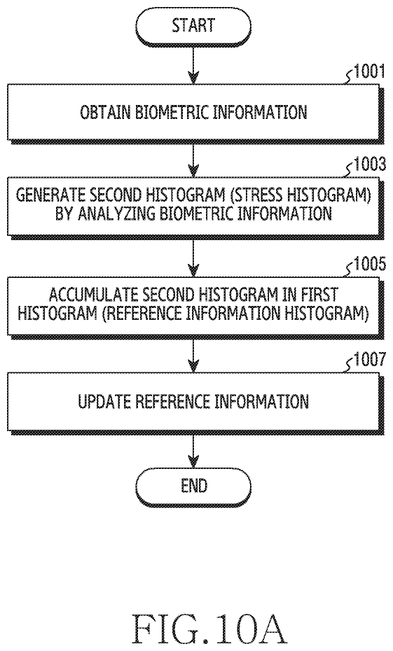

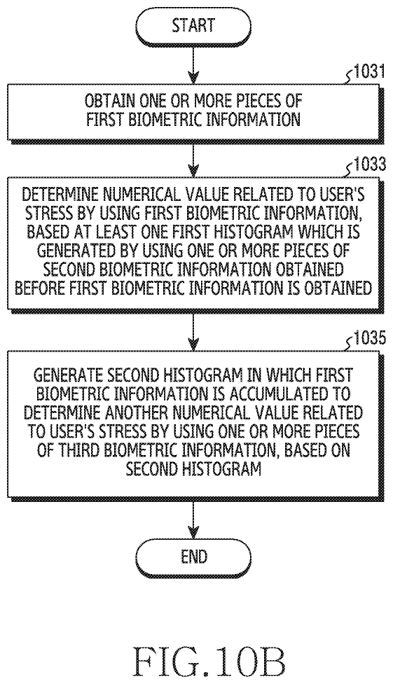

In embodiments of the disclosure, the reference information can be updated by reflecting a change in the cardiovascular characteristics of the user over time, and the stress can be exactly measured. A procedure of updating the reference information will be described in detail hereinbelow with reference to FIG. 10.

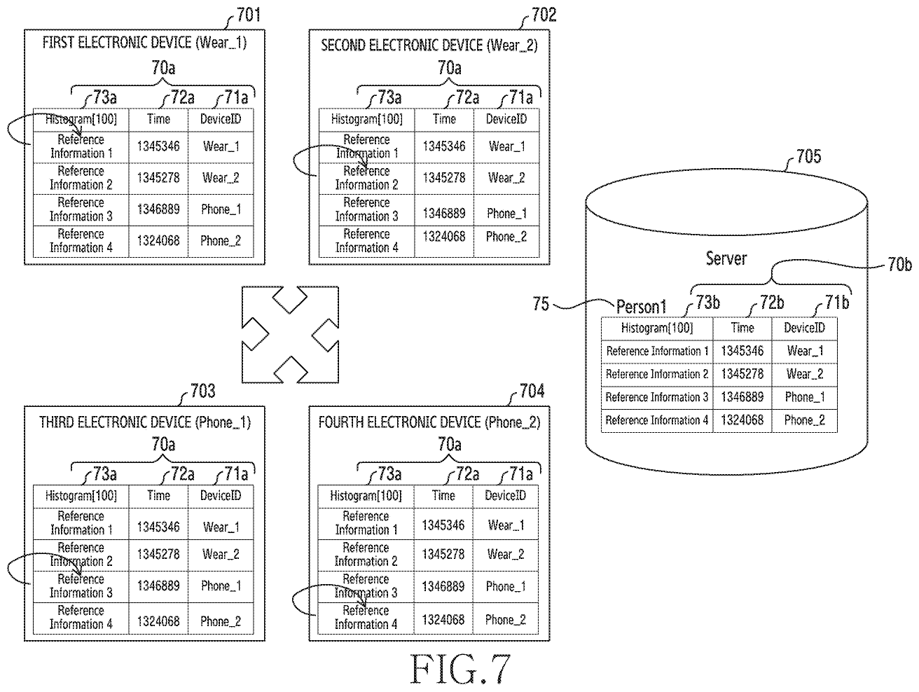

The processor 310 according to an embodiment of the disclosure may share the reference information and/or the reference information histogram with at least one external electronic device. The at least one external electronic device may be an electronic device that is owned by the user. According to an embodiment, the electronic device 301 and the at least one external electronic device may share the reference information histogram through a server. The reference information histogram is shared such that user's own electronic devices can provide the same measurement result to the user in the same environment. In other words, in embodiments of the disclosure, a measurement deviation that may occur between a plurality of electronic devices in the same environment due to different reference information can be prevented. Sharing the reference information histogram will be described in detail with reference to FIG. 7.

The processor 310 according to an embodiment of the disclosure may calibrate HRV data obtained through the biometric sensor 340. For example, the processor 310 may calibrate the measured HRV data by using interpolation. This is to compensate for a deviation caused by a difference in specification (performance) of biometric sensors. For example, when the biometric sensor 340 measures an HRV with a scan frequency of a first value (for example, 20 Hz), the processor 310 may calibrate heart rate data to correspond to heart rate data measured with a scan frequency of a second value (for example, 100 Hz) by using various well-known interpolation methods. Herein, the second value may be a scan frequency of a biometric sensor that has the best performance from among the plurality of electronic devices owned by the user. To achieve this, the plurality of electronic devices may share the performance of the biometric sensor with one another. Alternatively, the second value may be a value that is designated by the user.

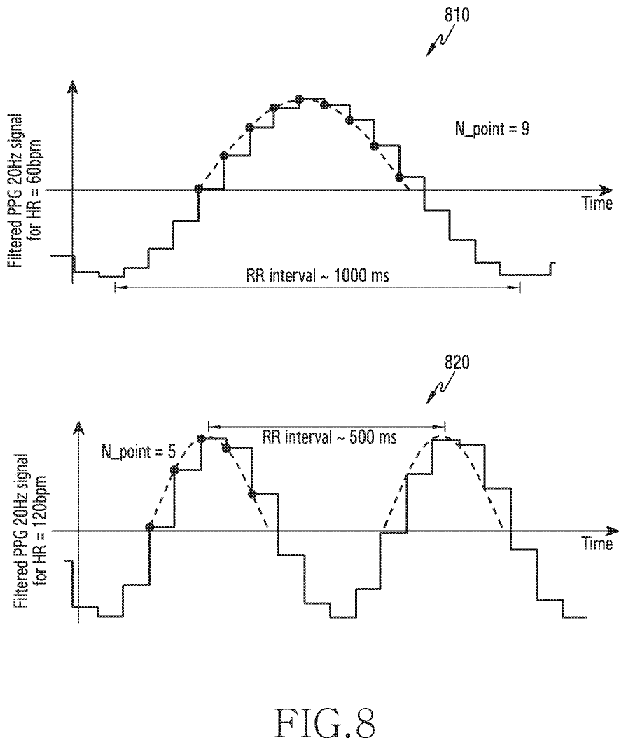

The processor 310 may change an interpolation rate according to a heart rate. For example, when the heart rate is a first value (for example, 120 ppm), the processor may interpolate the measured heart rate data by using N windows (for example, five windows), and, when the heart rate is ½ of the first value (for example, 60 ppm), the processor may interpolate the measured heart rate data by using 2N−1 (for example, nine) windows. This will be described in detail hereinbelow with reference to FIG. 8.

The memory 320 (for example, the memory 130) may be positioned inside a housing of the electronic device 301, and may be electrically (or functionally) connected with the processor 310. The memory 320 may store various programs and may store data which is generated or downloaded while the various programs are executed. The memory 320 may store various commands and/or instructions for operating the processor 310. The memory 320 may include at least one of an internal memory or an external memory.

According to various embodiments, the memory 320 may store a program that causes the processor 310 to perform various operations related to updating and sharing of the reference information histogram of the electronic device 301. The memory 320 may store the reference information histogram.

The display 330 (for example, the display device 160) may be exposed through a first surface of the housing of the electronic device 301, and may provide an output function. For example, the display 330 may be formed with a liquid crystal display (LCD), a light emitting diode (LED) display, an organic LED (OLED) display, or a micro electro mechanical system (MEMS) display, or an electronic paper display. According to an embodiment, the display 330 may include a touch panel to receive a user input. The touch panel may include a first panel (not shown) to detect a touch by a finger, a second panel (not shown) to recognize an input by an electronic pen, and/or a third panel (not shown) to detect a pressure.

According to various embodiments of the disclosure, the display 330 may display a stress measurement result. For example, the display 330 may display a stress measurement result screen as shown in FIG. 6. The result screen may include measurement date and time information 601, current stress information 602, average stress information 603, current heart rate information 604, oxygen saturation information 605, and a measurement start menu 606. The result screen of FIG. 6 is merely an example and does not limit embodiments of the disclosure. For example, the result screen may include some pieces of the information 601-605 or may further include other information.

The biometric sensor 340 (for example, the sensor module 176) may measure an HRV of the user according to a user's request or at set intervals. The biometric sensor 340 may be an electrocardiogram (ECG) sensor or a photoplethysmogram (PPG) sensor. According to an embodiment, the HRV may be measured by using a camera (not shown) and a flash (not shown) embedded in the electronic device 301.

The communication module 350 (for example, the communication module 190) may be positioned inside the housing of the electronic device 301 and may perform wired communication and/or wireless communication. For example, the communication module 350 may include at least one wireless (for example, mobile communication, WiFi and/or Bluetooth) communication circuit and/or at least one wired (for example, high definition multimedia interface (HDMI), display port (DP), or universal serial bus (USB), etc.) communication circuit.

According to various embodiments, the communication module 350 may transmit the reference information histogram to at least one external electronic device or server, or may receive a reference information histogram from the at least one external electronic device or server.

Although not shown in FIG. 3, some of the components described above may not be included in the electronic device 301. In another embodiment, the electronic device 301 may further include one or more other components (for example, a digital broadcasting module, a fingerprint recognition sensor, an input device, a memory, or the like) which are equivalent to the above-described components.

An electronic device (for example, the electronic device 101, 201, 202, 203, 301) according to various embodiments of the disclosure may include: a memory (for example, the memory 130, 320) configured to store a first histogram which is used to determine reference information for calculating a health condition of a user; a biometric sensor (for example, the sensor module 176, the biometric sensor 340); and at least one processor (for example, the processor 120, 210) operatively connected with the memory and the biometric sensor, and the at least one processor may be configured to: obtain biometric information through the biometric sensor; generate a second histogram by analyzing the obtained biometric information; update the first histogram by accumulating the second histogram in the first histogram; and update the reference information based on the updated first histogram.

According to various embodiments, the reference information may be a median of the first histogram.

According to various embodiments, the first histogram may be measured in a resting state of the user.

According to various embodiments, the at least one processor may be configured to multiply the first histogram by a forgetting factor, and to accumulate the second histogram.

According to various embodiments, the biometric sensor may include a heart rate measurement sensor configured to measure an HRV.

According to various embodiments, the electronic device may further include a display (for example, the display device 160, the display 330), and the at least one processor may be configured to analyze the HRV and to calculate a stress based on the updated reference information, and to display the calculated stress on the display.

According to various embodiments, the electronic device may further include a communication module (for example, the communication module 190, the communication module 350), and the at least one processor may be configured to share the updated first histogram with a least one of at least one other electronic device and a server connected through the communication module.

According to various embodiments, the at least one processor may be configured to calibrate the obtained biometric information by using an interpolation method.

According to various embodiments, the at least one processor may be configured to vary an interpolation rate according to a heart rate.

According to various embodiments, the at least one processor may be configured to provide a warning message to the user when the calculated stress is greater than or equal to a set value.

An electronic device (for example, the electronic device 101, 201, 202, 203, 301) according to various embodiments of the disclosure may include: a display (for example, the display device 160, the display 330); a biometric sensor (for example, the sensor module 176, the biometric sensor 340); and a processor operatively connected with the display and the biometric sensor, and the processor (for example, the processor 120, 210) may be configured to: obtain one or more pieces of first biometric information through the biometric sensor; determine a numerical value related to a user's stress by using the one or more pieces of first biometric information, based at least on a first histogram which is generated according to a frequency corresponding to a change of a period during which one or more pieces of second biometric information obtained before the one or more pieces of first biometric information are obtained are measured, and, based at least on the determination, generate a second histogram in which a frequency corresponding to a change of a period during which the one or more pieces of first biometric information are measured is accumulated, to determine another numerical value related to the user's stress by using one or more pieces of third biometric information to be measured from the user based at least on the second histogram.

According to various embodiments, the processor may be configured to obtain the first biometric information until a designated condition is satisfied.

According to various embodiments, the processor may be configured to determine the numerical value based at least on a reference value of the first histogram.

FIG. 7 is a view to explain a method for sharing a reference information histogram according to an embodiment of the disclosure.

Referring to FIG. 7, according to an embodiment of the disclosure, a user may own a plurality of electronic devices 701, 702, 703, 704 including a stress measurement function. The plurality of electronic devices 701-704 may share a reference information table 70a. In the reference information table 70a, device IDs 71a, generation times 72a, and reference information 73a may be mapped onto one another. Each of the electronic devices may update only the reference information corresponding to their own device IDs, and may synchronize the reference information table 70a by sharing the updated reference information and update time with other electronic devices through wired communication or wireless communication.

According to an embodiment, the electronic devices 701-704 may synchronize a reference information table 70b through a server 705. For example, the electronic devices 701-704 may transmit their own reference information, update times, and device IDs to the server 705. The server 705 may generate the reference information table 70b by combining the information received from the electronic devices 701-704, and may transmit the generated reference information table 70b to the respective electronic devices.

The server 705 may manage the reference information table 70b based on a user account 75. When the reference information table 70b is managed based on the user account 75, the user may receive the reference information table 70b from the server 705 through a user's account information input, and may store the reference information table 70b in a new electronic device. Accordingly, in various embodiments of the disclosure, even when the user uses a new electronic device (or initialized existing electronic device), the user can exactly measure a stress without going through an initial procedure of generating a reference information histogram.

Alternatively, when the user measures a stress by using an electronic device of the other person, not by the electronic device owned by the user, the user can measure the stress by using the reference information table 70b stored in the server 705 simply through an input of account information.

FIG. 8 is a view to illustrate a method for interpolating a heart rate measurement result according to an embodiment of the disclosure.

Referring to FIG. 8, according to an embodiment of the disclosure, heart rate measurement sensors included in a plurality of electronic devices (for example, the first electronic device 701 to the fourth electronic device 704 of FIG. 7) owned by a user may have different performance. For example, the heart rate measurement sensors of the first and second electronic devices 701, 702 may have a scan frequency of 20 Hz, and the heart rate measurement sensors of the third and fourth electronic devices 703, 704 may have a scan frequency of 100 Hz. When the scan frequencies of the heart rate measurement sensors are different as described above, graphs of HRV may be different in the same circumstance.

In an embodiment of the disclosure, heart rate data of a heart rate measurement sensor of relatively low performance may be calibrated to correspond to heart rate data of a heart rate measurement sensor of high performance by using various well-known interpolation methods (for example, parabolic interpolation). For example, the heart rate measurement sensor having the scan frequency of 20 Hz may obtain heart rate data at intervals of 50 (= 1/20) ms, and the heart rate measurement sensor having the scan frequency of 100 Hz may obtain heart rate data at intervals of 10 (= 1/100) m/s. In this case, the electronic device may calibrate the heart rate data obtained by the heart rate measurement sensor having the scan frequency of 20 Hz by interpolating four data between the heart rates on a 10 ms basis to have the same effect as the heart rate data is obtained at intervals of 10 ms like the heart rate measurement sensor having the scan frequency of 100 Hz. Through this, in embodiments of the disclosure, a measurement deviation caused by a difference in performance of the heart rate measurement sensors can be prevented by using interpolation.

According to an embodiment, the electronic device may vary an interpolation rate according to a heart rate. This is because the number of samples varies according to a human's heart rate. For example, the electronic device may use adaptive interpolation.

Referring to the drawing indicated by reference numeral 810 of FIG. 8, when a heart rate is 60 bpm, RRi may be about 1000 (=60 (seconds)/60) ms. In this case, a heart rate measurement sensor having a scan frequency of 20 Hz may obtain heart rate data at intervals of 50 (= 1/20) ms. In this case, the electronic device may generate a heart rate change graph by connecting nine (9) sampling points (N point) corresponding to a half period on the upper end of the horizontal axis (time axis) of an HRV graph.

Referring to the drawing indicated by reference numeral 820 of FIG. 8, when the heart rate increases to 120 bpm, RRi may be reduced to about 500 (=60 (seconds)/120) ms. In this case, the heart rate measurement sensor having the scan frequency of 20 Hz may obtain heart rate data at intervals of 50 (= 1/20) ms. When the period is reduced due to the increase of the heart rate, the electronic device may generate a heart rate change graph by connecting five (5) sampling points (N point) corresponding to a half period of the horizontal axis (time axis) of the HRV graph, and may. As described above, the electronic device according to an embodiment of the disclosure may change the number of samplings according to a heart rate.

FIG. 9 is a sequence diagram to explain a method for generating a reference information histogram of an electronic device according to an embodiment of the disclosure.

Referring to FIG. 9, a processor (for example, the processor 120, the processor 310) of the electronic device (for example, the electronic device 101, the electronic device 200, the electronic device 301) according to various embodiments of the disclosure may obtain biometric information in operation 901. For example, the processor may obtain heart rate data indicating a change in the heart rate through a biometric sensor (for example, the biometric sensor 340) in a resting state of a user.

In operation 903, the processor according to an embodiment of the disclosure may calculate a difference dRRi of intervals RRi between the heart rates based on the biometric information. For example, the processor may calculate a difference of continuous RRi.

In operation 905, the processor according to an embodiment of the disclosure may generate a histogram (for example, a reference information histogram) by accumulating dRRi based on sizes.

In operation 907, the electronic device according to an embodiment of the disclosure may determine whether the number of accumulated dRRi is greater than or equal to a set value (for example, 13).

When the number of accumulated dRRi is not greater than or equal to the set value as a result of identifying in operation 907, the processor may resume operation 901 to repeat the above-described operations. On the other hand, when the number of accumulated dRRi is greater than or equal to the set value as a result of identifying in operation 907, the processor may define reference information based on the histogram in which dRRi are accumulated by the set value or more. The reference information may be defined by a median of the reference information histogram.

Generating the reference information histogram and defining (setting) the reference information may be performed once initially. According to an embodiment, the procedure of generating the reference information histogram and defining the reference information may be re-performed upon receiving a user request or at set intervals.

FIG. 10A is a sequence diagram to explain a method for updating reference information according to an embodiment of the disclosure.

Prior to describing in detail, it is assumed that reference information for calculating a numerical value related to a stress is pre-stored through the method described in FIG. 9.

Referring to FIG. 10A, a processor (for example, the processor 120, the processor 310) of an electronic device (for example, the electronic device 101, the electronic device 200, the electronic device 301) according to various embodiments of the disclosure may obtain biometric information in operation 1001. For example, the processor may obtain heart rate data through a biometric sensor (for example, the biometric sensor 340) upon receiving a request of a user or at predetermined intervals.

In operation 1003, the processor according to an embodiment of the disclosure may generate a stress histogram by analyzing the biometric information. For example, the processor may generate the stress histogram by calculating RRi based on heart rate data, calculating dRRi based on the calculated RRi, and accumulating the calculated dRRi according to sizes.

In operation 1005, the processor according to an embodiment of the disclosure may update a reference information histogram by accumulating the stress histogram in the reference information histogram pre-stored in a memory (for example, the memory 130, the memory 320). According to an embodiment, the processor may update the reference information histogram by multiplying the reference information histogram by a forgetting factor and then accumulating the stress histogram. A value of the forgetting factor may vary in proportion to time.

In operation 1007, the processor according to an embodiment of the disclosure may update the reference information based on the updated reference information histogram. For example, the processor may calculate a median from the updated reference information histogram, and may update the reference information to the calculated median. The updated reference information may be used to calculate a stress afterward.

In the above-described embodiments of the disclosure, the reference information can be updated by reflecting a change in the cardiovascular characteristics which change with time, such that a stress can be exactly measured.

FIG. 10B is a flowchart to explain a method for updating reference information according to an embodiment of the disclosure.

Referring to FIG. 10B, a processor (for example, the processor 120, the processor 310) of an electronic device (for example, the electronic device 101, the electronic device 200, the electronic device 301) according to various embodiments of the disclosure may obtain one or more pieces of biometric information in operation 1031. For example, the processor may obtain first biometric information (for example, heart rate data) through a biometric sensor (for example, the biometric sensor 340) upon receiving a request of a user or at predetermined intervals. Operation 1031 may be performed until the first biometric information satisfies a designated condition (for example, a condition in which the number of dRRi generated based on the first biometric information is greater than or equal to a set value, or a designated time).

In operation 1033, the processor according to an embodiment of the disclosure may determine a numerical value related to a user's stress by using the first biometric information, based at least on a first histogram which is generated by using one or more pieces of second biometric information obtained before the first biometric information is obtained. The first histogram may be generated according to a frequency corresponding to a change of a period during which one or more pieces of second biometric information obtained before the first biometric information is obtained are measured. The first histogram may be stored in a memory (for example, the memory 130, the memory 320) of the electronic device or a server (for example, the server 108), and may be shared (or synchronized) with at least one other electronic device.

According to an embodiment, the processor may determine the numerical value related to the user's stress based at least on a reference value (reference information) of the first histogram.

In operation 1035, the processor according to an embodiment of the disclosure may generate a second histogram in which the first biometric information is accumulated to determine another numerical value related to the user's stress by using one or more pieces of third biometric information, based least on the second histogram. The second histogram may be generated by accumulating a frequency corresponding to a change of a period during which one or more pieces of first biometric information are measured, in the first histogram. According to an embodiment, the processor may generate the second histogram by multiplying the first histogram by a forgetting factor and then accumulating the first biometric information. The forgetting factor may vary in proportion to time.

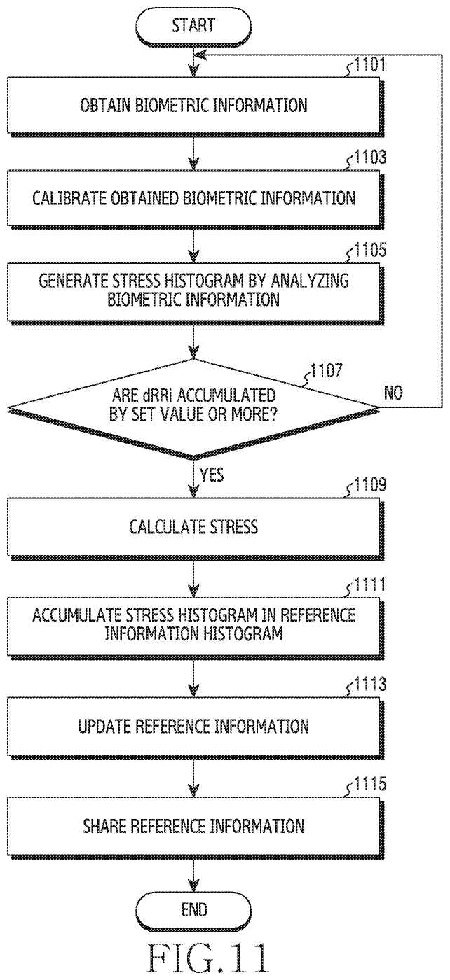

FIG. 11 is a flowchart to explain a method for measuring a stress of an electronic device according to an embodiment of the disclosure.

Prior to describing in detail, it is assumed that reference information for calculating a numerical value related to a stress is pre-stored through the method described in FIG. 9.

Referring to FIG. 11, a processor (for example, the processor 120, the processor 130) of the electronic device (for example, the electronic device 101, the electronic device 200, the electronic device 301) according to various embodiments of the disclosure may obtain biometric information in operation 1101. For example, the processor may obtain the biometric information (for example, an HRV) through a biometric sensor (for example, the biometric sensor 340) upon receiving a request of a user or at predetermined intervals.

In operation 1103, the processor according to an embodiment of the disclosure may calibrate the obtained biometric information. For example, the processor may calibrate the obtained biometric information by using interpolation. This is to prevent a deviation caused by a difference in performance of biometric sensors. According to an embodiment, the processor may calibrate the biometric information by using adaptive interpolation which changes the number of samplings according to a heart rate. This operation has been described above in FIG. 8 and thus a detailed description thereof is omitted. According to an embodiment, operation 1103 may be omitted.

In operation 1105, the processor according to an embodiment of the disclosure may generate a stress histogram by analyzing the biometric information (for example, obtained heart rate data or calibrated heart rate data). For example, the processor may generate the stress histogram by calculating RRi based on the heart rate data, calculating dRRi based on the calculated RRi, and accumulating the calculated dRRi according to sizes.

In operation 1107, the processor according to an embodiment of the disclosure may identify whether the number of accumulated dRRi is greater than or equal to a set value (for example, 13). When the number of accumulated dRRi is not greater than or equal to the set value as a result of identifying in operation 1107, the processor may resume operation 1101. On the other hand, when the number of accumulated dRRi is greater than or equal to the set value as a result of identifying in operation 1107, the processor may calculate a stress in operation 1109. For example, the processor may calculate a ratio of the total number of dRRi accumulated in the stress histogram and the number of dRRi having values less than or equal to previously set reference information.

According to an embodiment, the processor may generate stress history information by recording the calculated stress. According to an embodiment, the processor may display the calculated stress on a display (for example, the display 160, the display 330) according to a user's request. According to an embodiment, when the measured stress is greater than or equal to a set value (for example, 90%), the processor may notify the stress to the user through at least one of sight, hearing, and/or touch without a user's request.

In operation 1111, the processor according to an embodiment of the disclosure may update a reference information histogram by accumulating the generated stress histogram in the reference information histogram. According to an embodiment, the processor may multiply the reference information histogram by a forgetting factor before accumulating the stress histogram.

In operation 1113, the processor according to an embodiment of the disclosure may update reference information based on the updated reference information histogram. For example, the processor may calculate a median from the updated reference information histogram, and may update the reference information to the calculated median. The updated reference information may be used to calculate a stress afterward.

In operation 1115, the processor according to an embodiment of the disclosure may share the updated reference information with an external electronic device or a server. For example, the processor may transmit the updated reference information to the external electronic device or server through a communication module (the communication module 190, the communication module 350).

According to an embodiment, the processor may receive reference information from the external electronic device or the server through the communication module, and may update the reference information.

Although FIG. 11 depicts that the reference information is updated after the stress is calculated, the processor according to an embodiment may calculate a stress based on updated reference information after updating the reference information. In other words, operation 1109 may be performed after operation 1113.

In the above-described embodiments of the disclosure, the reference information can be updated by reflecting a change in the cardiovascular characteristics of the user which change with time, such that a stress can be exactly measured. In addition, in embodiments of the disclosure, the reference information is shared by the plurality of electronic devices, such that a problem that measurement results are different due to different reference information among the electronic devices can be prevented. In addition, in embodiments of the disclosure, a measurement result of a biometric sensor of low performance is calibrated to correspond to a biometric sensor of high performance, such that a measurement deviation caused by a difference in the performance between sensors can be reduced.

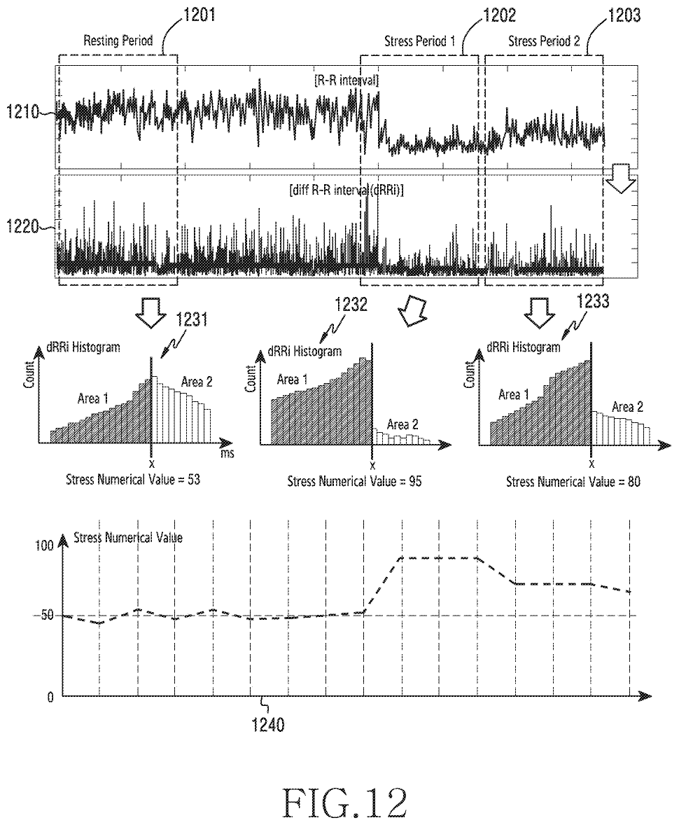

FIG. 12 is a view illustrating an example of stress measurement of an electronic device according to an embodiment of the disclosure.

Referring to FIG. 12, the electronic device according to an embodiment of the disclosure may measure a change of a heart rate (HRV) of a user by using a hear rate measurement sensor. Reference numeral 1210 of FIG. 12 is an HRV graph indicating a change of a heart rate. Reference numeral 1220 of FIG. 12 is a dRRi graph of the HRV graph 1210.

Referring to the HRV graph 1210 and the dRRi graph 1220, the HRV is different in a resting period 1201 and stress periods 1202, 1203.

The electronic device may generate a histogram by accumulating dRRi. Reference numeral 1231 indicates a dRRi histogram of the resting period 1201, reference numeral 1232 indicates a dRRi histogram of the first stress period 1202, and reference numeral 1233 indicates a dRRi histogram of the second stress period 1203. Referring to the histograms 1231, 1232, 1233, it can be seen that a numerical value related to the stress in the resting period 1201 is 53, a numerical value related to the stress in the first stress period 1202 is 95, and a numerical value related to the stress in the second stress period 1203 is 80.

Reference numeral 1240 of FIG. 12 indicates a stress history graph which continuously records a change of the stress. Referring to graphs of FIG. 12, it can be seen that the electronic device according to embodiments of the disclosure exactly measures a user's stress.

Table 1 presented below shows a result of an experiment which compares HRVs measured when a plurality of users rest (baseline), give presentations, and ask questions by using the stress measurement algorithm according to an embodiment of the disclosure and other stress measurement algorithms.

TABLE 1
One-Way ANOVA Test

| | Corre- | p-value | |
|---|---|---|---|
| Feature | spondence | 30 sec window | 10 sec window |
| Personalized | Baseline vs | 1.284E−09*** | 1.434E−09*** |
| pNNx | Presentation | | |
| | Baseline vs | 2.121E−05*** | 2.267E−05*** |
| | Question | | |
| | Presentation | 3.949E−02* | 4.601E−02* |
| | vs | | |
| | Question | | |
| pNN40- | Baseline vs | 1.198E−02* | 1.688E−02* |
| Median | Presentation | | |
| | Baseline vs | 1.886E−01 | 1.702E−01 |
| | Question | | |
| | Presentation | 4.799E−01 | 5.891E−01 |
| | vs | | |
| | Question | | |
| pNN10- | Baseline vs | 1.872E−03** | 3.252E−03** |
| Median | Presentation | | |
| | Baseline vs | 1.198E−01 | 1.307E−01 |
| | Question | | |
| | Presentation | 2.815E−01 | 3.437E−01 |
| | vs | | |
| | Question | | |
| SDDN-Median | Baseline vs | 3.714E−02* | 1.636E−01 |
| | Presentation | | |
| | Baseline vs | 8.310E−01 | 9.839E−01 |
| | Question | | |
| | Presentation | 1.385E−01 | 2.245E−01 |
| | vs | | |
| | Question | | |
| RMSSD- | Baseline vs | 1.201E−02* | 1.882E−02* |
| Median | Presentation | | |
| | Baseline vs | 2.824E−01 | 4.263E−01 |
| | Question | | |
| | Presentation | 3.496E−01 | 2.920E−01 |
| | vs | | |
| | Question | | |

Referring to table 1 described above, the probability value (p-value) refers to a probability that compared data is independent. For example, as the probability value decreases, comparison data may be data having no relevance. In addition, the window means a measurement time. For example, the 30 second window means that heart rate data is measured for 30 seconds, and the 10 second window means that heart rate data is measured for 10 seconds.

Referring to the result of comparing heart rate data, measured when the user rests (baseline) and gives a presentation, by the personalized pNNx algorithm according to an embodiment of the disclosure, it can be seen that, when the 30-second window is used, the probability value is 1.284E-09, and, when the 10-second window is used, the probability value is 1.434E-09. In addition, referring the result of comparing heart rate data measured when the user rests (baseline) and asks a question, it can be seen that, when the 30-second window is used, the probability value is 2.121E-05, and, when the 10-second window is used, the probability value is 2.267E-05. In addition, referring the result of comparing heart rate data measured when the user gives a presentation and asks a question, it can be seen that, when the 30-second window is used, the probability value is 3.949E-02, and, when the 10-second window is used, the probability value is 4.601E-02. Referring to probability values of other algorithms, it can be seen that the personalized pNNx algorithm according to embodiments of the disclosure can more clearly identify heart rate data measured when the uses rests, gives a presentation, and asks a question.

As described above, the result of measurement according to the personalized pNNx algorithm according to an embodiment of the disclosure is more exact than the results of measurement according to other algorithms (pNN40-Median, pNN10-Median, SDNN-Median, RMSSD-Median), and exact measurement is possible even when the 10-second window is used. Various embodiments of the disclosure as described above can reduce the time required to measure and thus can enhance user's convenience.

In other words, the electronic device according to an embodiment of the disclosure can measure a stress exactly and rapidly.

A stress measurement method of an electronic device which stores a first histogram which is used to determine reference information for calculating a health condition of a user according to various embodiments may include: obtaining biometric information through a biometric sensor; generating a second histogram by analyzing the obtained biometric information; updating the first histogram by accumulating the second histogram in the first histogram; and updating the reference information based on the updated first histogram.

According to various embodiments, the reference information may be a median of the first histogram.

According to various embodiments, the first histogram may be measured in a resting state of the user.

According to various embodiments, updating the first histogram may include multiplying the first histogram by a forgetting factor.

According to various embodiments, obtaining the biometric information may include obtaining an HRV.

According to various embodiments, the method may further include analyzing the HRV and calculating a stress based on the updated reference information; and providing the calculated stress.

According to various embodiments, the method may further include sharing the updated first histogram with at least one of at least one electronic device and a server.

According to various embodiments, obtaining the biometric information may further include interpolating the obtained biometric information.

According to various embodiments, interpolating may include varying an interpolation rate according to a heart rate.

According to various embodiments, the method may further include providing a warning message when the calculated stress is greater than or equal to a set value.

The electronic device according to various embodiments may be one of various types of electronic devices. The electronic devices may include, for example, at least one of a portable communication device (e.g., a smart phone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, and/or replacements for a corresponding embodiment. With regard to the description of the drawings, similar reference numerals may be used to refer to similar elements. It is to be understood that a singular form of a noun may include one or more of the things, unless the relevant context clearly indicates otherwise. As used herein, each of such phrases as “A or B,” “at least one of A and/or B,” “A, B, or C,” or “at least one of A, B, and/or C” may include all possible combinations of the items enumerated together. As used herein, such terms as “1st,” “2nd,” “first” or “second” may modify corresponding components regardless of an importance or an order, be used to distinguish a component from another, and does not limit the corresponding components. It is to be understood that if an element (e.g., a first element) is referred to, “(operatively or communicatively) connected with,” or “connected to” another element (e.g., a second element), it means that the element may be coupled with the other element directly, or via other element (e.g., a third element).

As used herein, the term “module” includes a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, the module may be implemented in a form of an application-specific integrated circuit (ASIC).

Various embodiments as set forth herein may be implemented as software (e.g., the program 140) including instructions that are stored in a machine readable storage medium (e.g., internal memory 136 or external memory 138) that is readable by a machine (e.g., computer). The machine may invoke instructions stored in the storage medium, be operated to perform functions according to the instructions invoked, and include the electronic device (e.g., the electronic device 101, the electronic device 201, the electronic device 301) according to embodiments disclosed. If the instructions are executed by a processor (e.g., the processor 120, the processor 310), the processor may execute functions corresponding to the instructions directly or using other components under the control of the processor. The instructions may include a code generated or executed by a compiler or an interpreter. The machine-readable storage medium may be provided in the form of a non-transitory storage medium. Wherein, the term “non-transitory” simply means that the storage medium does not include a signal and is tangible, but does not differentiate between semi-permanently storing the data in the storage medium and temporarily storing the data in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed online via an application store (e.g., Play Store™). If distributed online, at least part of the computer program product may be temporarily generated or at least temporarily stored in the storage medium, such as memory of the manufacturer's server, a server of the application store, or a relay server.

According to various embodiments, each component (e.g., a module or a program) may include a single entity or multiple entities, and part of the above-described components may be omitted, or other components may be added. Alternatively or additionally, the part of components (e.g., modules or programs) may be integrated into a single component, and may still perform a function of each component in the same or similar manner as they are performed by each component before the integration. According to various embodiments, operations performed by the module, the program, or another component may be carried out sequentially, in parallel, repeatedly, or heuristically, or at least part operation may be executed in a different order or omitted, or other operations may be added.

1. An electronic device comprising:
a memory configured to store a first histogram which is used to determine reference information for calculating a health condition of a user;
a biometric sensor; and
at least one processor operatively connected with the memory and the biometric sensor,
wherein the at least one processor is configured to:
obtain biometric information through the biometric sensor;
generate a second histogram by analyzing the obtained biometric information;
update the first histogram by accumulating the second histogram in the first histogram; and
update the reference information based on the updated first histogram.

2. The electronic device of claim 1, wherein the reference information is a median of the first histogram.

3. The electronic device of claim 1, wherein the first histogram is measured in a resting state of the user.

4. The electronic device of claim 1, wherein the at least one processor is configured to multiply the first histogram by a forgetting factor, and to accumulate the second histogram.

5. The electronic device of claim 1, wherein the biometric sensor comprises a heart rate measurement sensor configured to measure an HRV.

6. The electronic device of claim 5, further comprising a display,
wherein the at least one processor is configured to analyze the HRV and to calculate a stress based on the updated reference information, and to display the calculated stress on the display.

7. The electronic device of claim 1, further comprising a communication module,
wherein the at least one processor is configured to share the updated first histogram with a least one of at least one other electronic device and a server connected through the communication module.

8. The electronic device of claim 1, wherein the at least one processor is configured to calibrate the obtained biometric information by using an interpolation method.

9. The electronic device of claim 8, wherein the at least one processor is configured to vary an interpolation rate according to a heart rate.

10. The electronic device of claim 6, wherein the at least one processor is configured to provide a warning message to the user when the calculated stress is greater than or equal to a set value.

11. A stress measurement method of an electronic device which stores a first histogram which is used to determine reference information for calculating a health condition of a user, the method comprising:
obtaining biometric information through a biometric sensor;
generating a second histogram by analyzing the obtained biometric information;
updating the first histogram by accumulating the second histogram in the first histogram; and
updating the reference information based on the updated first histogram.

12. The method of claim 11, wherein the reference information is a median of the first histogram.

13. The method of claim 11, wherein updating the first histogram comprises multiplying the first histogram by a forgetting factor.

14. The method of claim 11, wherein obtaining the biometric information comprises obtaining an HRV,
the method further comprising:
analyzing the HRV and calculating a stress based on the updated reference information;
providing the calculated stress; and
providing a warning message when the calculated stress is greater than or equal to a set value.

15. The method of claim 11, wherein obtaining the biometric information further comprises interpolating the obtained biometric information, and
wherein interpolating comprises varying an interpolation rate according to a heart rate.

16. The method of claim 11, wherein the reference information is a median of the first histogram.

17. The method of claim 11, further comprising sharing the updated first histogram with a least one of at least one other electronic device and a server.

18. An electronic device comprising:
a display;
a biometric sensor; and
a processor operatively connected with the display and the biometric sensor,
wherein the processor is configured to:
obtain one or more pieces of first biometric information through the biometric sensor;
determine a numerical value related to a user's stress by using the one or more pieces of first biometric information, based at least on a first histogram which is generated according to a frequency corresponding to a change of a period during which one or more pieces of second biometric information obtained before the one or more pieces of first biometric information are obtained are measured, and
based at least on the determination, generate a second histogram in which a frequency corresponding to a change of a period during which the one or more pieces of first biometric information are measured is accumulated, to determine another numerical value related to the user's stress by using one or more pieces of third biometric information to be measured from the user based at least on the second histogram.

19. The electronic device of claim 18, wherein the processor is configured to obtain the first biometric information until a designated condition is satisfied.

20. The electronic device of claim 18, wherein the processor is configured to determine the numerical value based at least on a reference value of the first histogram.
